(19) United States
(12) Patent Application Publication
(10) Pub. No.: US 2023/0226295 A1
(43) Pub. Date: Jul. 20, 2023

(54) INTUBATION AID

(71) Applicant: Reiner KUNZ, Kleinmachnow (DE)

(72) Inventors: Reiner KUNZ, Kleinmachnow (DE); Michael SCHMIDT, Halifax, Nova Scotia (CA)

(73) Assignee: Reiner KUNZ, Kleinmachnow (DE)

(21) Appl. No.: 17/923,271

(22) PCT Filed: Apr. 27, 2021
(86) PCT No.: PCT/EP2021/060902

§ 371 Date: Nov. 4, 2022

(30) Foreign Application Priority Data
May 6, 2020 (DE) 10 2020 205 723.0

(51) Int. Cl. A61M 16/04; A61M 16/10; A61B 1/267

(52) U.S. Cl. CPC A61M 16/0497; A61M 16/1005; A61M 16/1055; A61B 1/267; A61M 2202/0208

(57) ABSTRACT

One aspect of the invention relates to an intubation aid (10). The intubation aid (10) comprises an elongated main part (12), and the intubation aid (10) is provided with an operating device (26). The main part (12) is designed to be curved in a first region (18) of the main part (12), and the main part (12) is additionally designed to be curved in a second region (20) of the main part (12), said second region being separated from the first region (18) and being arranged adjacently to the free end (16) of the main part (12). The main part (12) additionally comprises a positioning device (24) which is designed to limit a translational displacement of a tube (46) placed on the intubation aid (10) in at least one direction. A second aspect of the invention relates to an intubation aid which can be placed on an endoscope.

The present invention relates to an intubation aid for inserting a tube into a patient's trachea.

Diagnostic and therapeutic interventions on humans and animals are often carried out under anaesthesia, with adequate breathing being ensured by inserting a breathing tube into the trachea. As a rule, the head is reclined in order to facilitate the introduction of the ventilation tube while the oral cavity/pharynx/larynx/trachea are aligned as straight as possible. The tongue is held to the side with an intubation spatula and the tube (ventilation tube) is advanced into the trachea under visual control and blocked there in order to achieve reversible, atraumatic, and airtight fixation on the mucous membrane of the trachea. In this way, even with increased ventilation pressure in the trachea and lungs, it is possible to avoid a collapse of the alveoli, also in order to optimize gas exchange.

For this purpose, the tube is often gripped directly by the operator and inserted into the patient. However, there is a risk of injury to the patient, since a completely straight alignment of the oral cavity/pharynx/larynx/trachea can never be achieved, but rather is in an S-shaped course, and the tube is only designed to be passively adapted to the anatomy of the patient (i.e. by sliding it on the patient's body structures).

There are attempts available on the market to attach the ventilation tube to a bronchoscope in order to be able to actively manipulate the tube using the mechanical features of the bronchoscope. However, bronchoscopes are too unwieldy because they are too long and also too thin for intubation, so that they often cannot sufficiently deform the very rigid tube or can even kink it. Furthermore, bronchoscopes are very expensive, so that they cannot be made available in the number required for the large number of intubations to be carried out, especially in the context of the COVID-19 pandemic.

It is therefore the object of the present invention to provide an intubation aid which can also ensure safe intubation as part of critical airway management.

This object is achieved in a first aspect according to the invention by an intubation aid for inserting a tube into a patient's trachea, wherein the intubation aid comprises an elongated main part which at its one end is connected to a handle and at its other end has a free end, wherein the intubation aid, in particular the handle, is provided with an operating device, wherein the main part is designed to be bent in a first region of the main part when the operating device is actuated, wherein the main part is furthermore designed, upon further actuation of the operating device, to be bent in a second region of the main part which is separate from the first region and is arranged adjacent to the free end of the main part, and wherein the main part further comprises a positioning device which is designed to limit a translational displacement of a tube fitted onto the intubation aid in at least one direction.

A “translational displacement” is to be understood here as a movement parallel to a longitudinal axis of the main part. In other words, it is a displacement of the tube in and opposite to the direction in which it is fitted over the intubation aid.

The positioning device can be designed, for example, as a stop against which one end of the tube can rest. The stop can be adjustable in particular relative to the main part such that the positioning device can be adapted or adjusted to different tube lengths. When using bronchoscopes, adhesive tape must currently be used to hold the tube on the bronchoscope.

It should also be mentioned at this point that “actuation of the operating device” in this aspect of the invention means that the main part or a tube arranged on the main part is not bent directly at the region to be bent (for example, manually), but that this bending takes place remotely, for example using a cable connected to the operating device. In this case, the operating device can be particularly suitable for being actuated by a user's thumb. By adjusting the bendable regions of the inventive intubation, the main part, and thus the tube arranged thereon, can be deformed such that they can be adapted to the anatomical situation of the patient during intubation, and thus the risk of injury, in particular to the larynx and vocal cords, is reduced or can even be completely avoided.

As an alternative or in addition to mechanical actuation of the first or second region to be curved using the operating device, input from an operator can also be recorded electronically on the operating device and output to servomotors which are assigned to the first region or second region and are designed to bring about a corresponding bend in the first region or second region.

For improved insertion of the intubation aid into the patient or guiding of the tube onto the intubation aid, at least the main part can be provided with a non-stick coating on its exterior.

In a further development of the present invention, the positioning device can comprise a holding device which is designed to engage with a segment of the tube in order to secure the latter, at least translationally, relative to the main part, wherein the holding device in particular is a cone that is arranged adjacent to the handle and that engages a segment of the tube in a friction fit, and/or is embodied as a locking unit which engages the segment of the tube in a positive fit. The expression “secure” is to be understood such that the holding device holds the tube on the main part until a release force predetermined by the holding device is exceeded in a direction for removing the tube from the main part. The holding device can include an active release functionality, both for securing in a friction fit and for securing in a positive fit, which can reduce the predetermined releasing force for removing the tube from the main part. This can be realized, for example, by reducing the radial extent of the above-mentioned cone and/or by moving the above-mentioned locking unit. In a possible embodiment of the holding device, a hook can engage behind an end segment of the tube in order to secure the tube on the main part. Of course, the holding device can be suitable for different tube diameters.

Advantageously, the main part can comprise a channel which extends parallel, at least in part, to a longitudinal axis of the main part and which is suitable for receiving fluid and conducting the fluid to an end of the channel assigned to the handle. For example, if a tube is fitted over the main part, the tube, together with the main part, can form a corresponding suction channel. It can be advantageous for the free cross-section of the tube, which can be used for suctioning off fluid, to be filled as minimally as possible by the main part.

For example, the pulmonary disease COVID-19 (Corona) is caused by SARS-CoV-2 viruses. These are mainly found in the mouth/nasal passages/pharynx and in the larynx and are transmitted from person to person through droplet infection and the formation of aerosol in the air we breathe. Droplets form when a person speaks, sings, coughs, sneezes, or breathes deeply and, depending on their size, sink to the ground. However, aerosols can linger in the air in closed spaces for hours, with a correspondingly longer possibility of infection. Since SARS-CoV-2 viruses are found in large quantities in the mucous membrane of the upper airways of those infected, and Corona patients shed the virus and are therefore infectious several days before the first symptoms appear, the pathogens and the disease can be passed on at any time and this is the reason this viral disease is so extremely dangerous. At least 80% of those infected have an asymptomatic course or only minor symptoms.

During an intubation or an endoscopy of the nose/pharynx or a bronchoscopy or gastroscopy, the examiner cannot maintain a safe distance from the sick person and is at risk of taking in the infectious material (bacteria/virus), also due to the maximally forced breathing of patients with COVID-19, and contracting the same disease. In order to protect medical staff from becoming infected by the virus, they wear protective suits, protective goggles, visor-like protective masks, and gloves, patients are covered with disposable foils and occasionally the upper body and head region are covered with polyacrylic boxes during intubation, the boxes being suctioned at >100 L/min to prevent the spread of the virus.

Continuous suctioning of the virus-laden air from the mouth/nasal passages/pharynx, larynx, and trachea of the patient during the intubation process can thus reduce or prevent contamination of the ambient air in the operating room and consequently can reduce or prevent the risk of infection for the treating staff.

For this purpose, a suction unit and a virus and bacteria filter, which is suitable for filtering viruses and bacteria from the fluid suctioned from the patient, or a gas source, in particular an oxygen source, which is designed to deliver oxygen to the patient via the channel, can be selectively connected to the end of the channel assigned to the handle. For example, a lip valve can be arranged at the end of the channel. A pressure measuring device can also be coupled to the channel in order to be able to detect an inhalation and exhalation phase of the patient, so that during the inhalation phase oxygen can be introduced into the patient's lungs and in the exhalation phase the aerosol which the patient would otherwise exhale can be diverted and filtered. To this end, it can be advantageous for the interface for suctioning off aerosol to be embodied separate from an interface for introducing oxygen.

Furthermore, a wall of the main part, viewed in the circumferential direction of the elongate main part, can be interrupted such that a gap extending in the longitudinal direction of the main part is formed and connects the channel to an exterior surrounding the main part. For example, a main part that has an essentially crescent-shaped section can result. This access to the channel from the exterior over at least a majority of the longitudinal extension of the channel can significantly improve the cleanability of the channel, since in particular cleaning utensils can be inserted into the channel from the side and/or sterilizing fluid can enter the channel from the side. In addition to the above-described channel for suctioning off the aerosol from the patient or for introducing gas, such as oxygen, the main part can of course include further channels which, in particular, can only open at their longitudinal ends to an exterior of the main part. For example, such a further channel can be suitable for inserting biopsy forceps.

In particular, the main part can have a tapering segment which is arranged adjacent to the free end of the main part, in particular between the free end of the main part and the second bendable region of the main part. This tapering segment can in particular be designed such that it is embodied to match an inner diameter of the tube and/or a length of the tube, that is, permits a gradual transition from a segment assigned to the free end of the main part to at least one inner diameter of the tube, in particular an outer diameter of the tube. For this purpose, the above-mentioned positioning device and/or holding device of the main part can be displaceable such that the end of the tube opposing the positioning device and/or holding device can be aligned with the tapering segment of the main part.

This gradual, that is, essentially not abrupt, transition of the free end of the main part to the tube can prevent the intubation aid or the tube from getting caught on the patient's vocal cords and the patient's vocal cords from being damaged when the tube is inserted. For example, the distance between the free end of the main part and the conically tapering (or, viewed from this direction, the widening) segment of the main part can be approximately 5 cm.

In an embodiment of the present invention, the channel may extend from the end associated with the handle to the tapering segment of the main part. That is, the channel terminates at the tapering segment of the main part or gradually runs radially outwards there. When the tube is arranged on the main part, this can enable the aerosol to be suctioned off directly at the distal end of the tube.

Advantageously, an imaging unit, in particular an image sensor, that is designed to record and forward image data, can be arranged at the free end of the main part. Video documentation of the intubation process means that there is no need to subsequently listen to both lungs with a stethoscope. In addition, the operator receives visual information about the alignment of the intubation aid and tube during intubation. In combination with the above-mentioned servomotors for bending the first segment or second segment, it is possible for the imaging unit to output the recorded image data to a control unit which analyses the image data (for example, with regard to a maximum distance of the imaging unit from an anatomical structure of the patient, which can indicate, for example, the further course of the patient's trachea) and based thereon, automatically control the servomotors (for example, align the free end of the main part in the direction of this greatest distance). It could thus be possible for the inventive intubation aid to deform autonomously, that is, without input into the operating device of the intubation aid, in order to adapt to an anatomical situation of the patient during intubation. Of course, it can be advantageous for the control unit to carry out a plausibility check for corresponding activations of the servomotors to be performed, for example in order to be able to prevent the intubation aid from carrying out an undesired deformation when it is inserted into the patient's vocal cords. For this purpose, the imaging unit can be embodied as a stereo camera which is designed to record a region from different angles, or the imaging unit can comprise another device for measuring distance.

Furthermore, the intubation aid can be equipped with a sensor device which is suitable for detecting a position/orientation of the intubation aid and/or a user touching the intubation aid, and for emitting corresponding signals, so that the intubation aid can be switched on and off on the basis of corresponding signals from the sensor device.

An illumination device designed to emit light, in particular at the region to be recorded by the imaging unit, can be arranged on the free end of the main part. A light source can be arranged directly at the distal end of the main part or the light to be emitted can be fed from an external light source into light guides via which the light can be emitted at the distal end of the main part.

In particular, the first bendable region can be arranged at a distance of 10 cm to 20 cm, in particular about 15 cm, from the free end of the main part. This arrangement of the first region can enable the main part or the tube to be adapted to the anatomy of the majority of patients.

Planes in which the main part can be bent in the first region and in the second region can also be essentially parallel to one another, in particular coincident. This means that the bend in the first region and the bend in the second region run in essentially the same radial direction and/or in an essentially opposing radial direction.

In an advantageous embodiment of the present invention, the first region can be bent from 0° to 30° and/or the second region can be bent from −60° to 45°. In the context of the present invention, 0° shall be a straight alignment of segments of the main part upstream and downstream of the specific bendable region, that is, an essentially straight longitudinal extension of the main part in relation to the respective region. A 30° bend in the first region refers to a 30° deflection of the segment of the main part that is arranged distal from the first region relative to the 0° orientation. Positive degrees (here without a mathematical operator) can be understood as extending to a first side, in particular the cranial side, and negative degrees (for example)−60° as extending to a second side, in particular the caudal side.

The operating device can comprise at least a first operating element and a second operating element, wherein the first operating element is designed for deflecting the first region of the main part and the second operating element is designed for deflecting the second region of the main part, and wherein the first operating element is assigned a latching device designed for preventing rearward movement of the first operating element, counter to the actuating direction thereof, upon actuation of the first operating element by at least a predetermined amount. The latching device can of course be embodied detachable in order to be able to move the corresponding operating element, and thus the associated bendable region, back to its original orientation. Alternatively or additionally, the latching device can also be activated, that is, the first region can be locked, by actuating a special operating element in the handle, in particular on a finger support surface embodied there. In this case, the second operating element can in particular not include such a latching device, so that a free forward and backward movement of the second region of the main part can remain assured.

The intubation aid can further include a communication module, in particular a wireless communication module, which is designed to communicate with an external unit, wherein the communication module is arranged in particular in the handle of the intubation aid. A wireless communication module can be designed, for example, for WLAN communication, LiFi communication, or Bluetooth communication. The external unit can be a communication interface which is connected to a monitor or is integral therewith in order to display the image data recorded by the imaging unit of the intubation aid.

In particular in the case of wireless communication between the inventive intubation aid and the external unit, both the intubation aid and the external unit, for example the monitor, can be provided with an integrated power supply. The integrated power supply can be embodied as an accumulator, which can be charged inductively, for example.

In order to ensure communication of the intubation aid with an associated monitor or to prevent undesired communication of an intubation aid from a first intubation aid monitor pair with a monitor from a second intubation aid monitor pair, communication between the communication module of the intubation aid and the associated external unit can be connected via an active coupling (e.g. by pressing appropriate buttons within a predetermined period of time).

The handle may include at least a single-finger receiving segment, embodied to receive an index finger of an operator, and a two-finger receiving segment, embodied to receive a middle finger and a ring finger of an operator, wherein the single-finger receiving segment is disposed relative to the main part on the side towards which the first region is deflected when it is bent. This configuration of the handle can improve handling in the normal usage situation of the inventive intubation aid. In particular, the handle can be arranged relative to the main part such that there is essentially a T-shape between the handle and the main part. A T-shape can be advantageous, because the patient lies on the operating table at about hip height of the anaesthetist during the introduction to the operation. In an emergency, the emergency physician usually kneels in front of the patient, i.e., the patient's mouth is approximately level with the emergency physician's kneecap. In both cases, the right hand of the person performing the intubation pushes the tube downward and, at the same time, away from the body of the doctor. This movement can be supported by the T-shape of the handle of the intubation aid.

A segment of the communication module, in particular for LiFi communication, can also be arranged on a segment of the handle arranged between the thumb and forefinger when the handle is gripped by an operator. This segment of the communications module can also protrude from the handle such that the web between the operator's thumb and forefinger can be arranged between this segment of the communication module and the handle.

It should also be added that it may also be possible to provide an intubation aid for inserting a tube into a patient's trachea, which intubation aid comprises an elongate main part, wherein the main part further comprises a positioning device which is designed to limit a translational displacement of a tube placed on the intubation aid in at least one direction, in particular in a translational direction relative to the main part, and wherein the elongate main part, in particular the segment of the positioning device thereof, can be connected to a superordinate suction device, under the effect of which fluid can be suctioned from a patient along the main part. For example, if a tube is fitted over the main part, the tube, together with the main part, can form a corresponding suction channel. It can be advantageous for the free cross-section of the tube, which can be used for suctioning off fluid, to be filled as minimally as possible by the main part.

With regard to this embodiment, reference is also made to all of the features, effects, and advantages described in relation to the intubation aid of the first and/or the second aspect of the present invention which can also be used here, and vice versa.

In a second aspect, which is based on the same object as the first aspect, wherein this object is solved alternatively in the second aspect, the present invention relates to an intubation aid for inserting a tube into the trachea of a patient, comprising an elongate main part, a tapering segment which is arranged on and connected to a longitudinal end of the main part, a connecting segment which is arranged on and connected to the other longitudinal end of the main part, wherein the connecting segment has a first engaging segment which in the radial direction is essentially inwardly oriented and is designed to engage with a superordinate assembly, in particular an endoscope, and has a second engagement segment which in the radial direction is essentially outwardly oriented and is designed to engage with the tube, and wherein both the connecting segment and the tapering segment have a receiving segment each, each of which is essentially embodied as a through-hole and is designed to receive a segment of the superordinate assembly, in particular the endoscope.

It should be mentioned at this point that all the features, effects, and advantages described for the intubation aid of the first aspect of the present invention can also be applied to the intubation aid of the second aspect, and vice versa.

The first engagement segment and/or the second engagement segment of the connecting segment can be designed in particular to engage with the superordinate assembly and/or the tube in a friction fit, so that, for example, the tube can be pushed onto the intubation aid until the above-mentioned collar of the tube is gripped by the cone of the positioning device or the holding device, as already mentioned above. For this purpose, the first and/or the second engagement segment of the connecting segment can have a coating, for example made of rubber, which promotes this frictional fit. Alternatively or in addition, a locking unit that engages in a positive fit can also be provided here, of course.

In the intubation aid of the second aspect of the present invention, the main part can be designed such that, before or after the tube has been fitted onto the intubation aid, manual force exerted by the operator can bend the main part in at least one radial direction, in particular only along a plane in which a corresponding bend extends. It is also possible for the intubation aid, or at least the main part of the intubation aid, to be delivered in a pre-bent configuration, so that when a tube is fitted over the pre-bent main part, the tube is already deformed in a desired manner thereby. In particular if the main part is not to be designed to be bent by the operator during use, it can be advantageous to provide the intubation aid or the main part in at least two different variants which differ in the extent of the existing bend of the main part. For this purpose, the main part can be designed, for example, as a metal rod which connects the tapering segment to the connecting segment, in particular at a peripheral region of these two segments. In this way, the “first region” of the intubation aid can be implemented according to the first aspect of the present invention without having to provide special operating elements and cable pulls for this purpose.

In terms of its longitudinal extension, the intubation aid or the main part of the intubation aid can be provided in standard lengths of available ventilation tubes and/or can be adjustable in its longitudinal extension across a predetermined range. This can be implemented, for example, using a design of the main part that is adjustable in length or using the displaceable positioning device mentioned in relation to the first aspect. Thus, with the intubation aid according to the second aspect of this invention, it can also be achieved that the tapering segment can be aligned with a distal end of the tube fitted over the main part in order to be able to avoid injuries to the patient's vocal cords.

The connecting segment can be embodied as an element that is manufactured separately from the main part and is detachably connected thereto. Thus, the connecting segment and/or the main part can be embodied as disposable items to be discarded after a single use. For this purpose, the end of the main part assigned to the connecting segment can be embodied with a suitable connecting structure, such as, for example, a retaining clip, which can be placed on a corresponding counter-connecting structure of the connecting segment.

It should be explicitly pointed out that the connecting segment and/or the tapering segment can be embodied with the above-mentioned channel for suctioning off aerosol, which can be diverted using a corresponding interface on the connecting segment, or oxygen can be supplied through the channel via this interface.

The receiving segment of the intubation aid can in particular be designed for a standard diameter of an available endoscope, for example, a flexible bronchoscope.

In terms of an exemplary embodiment, the intubation aid of the second aspect of the invention can be fitted onto a standard endoscope, that is, the standard endoscope can be passed through the receiving segments of the connecting segment and of the tapering segment until the first engaging segment engages with a corresponding countersegment of the endoscope. A ventilation tube is then fitted onto the main part, which has already been preformed, for example. The free end of the endoscope, which usually includes a mechanism for bending the free end, can then protrude beyond the tapering segment of the intubation aid such that it satisfies the function of the “second region” and the “free end” of the intubation aid according to the first aspect of this invention.

It should also be pointed out at this point that the intubation aid according to the first aspect of the present invention and/or the intubation aid according to the second aspect of the present invention can comprise at least one intermediate piece which is designed to be arranged between the tube and the main part in order to be able to provide an adapter between the outside diameter of the intubation aid and the inside diameter of a specific tube in a simple manner. It can be advantageous for the intubation aid according to the first aspect of the present invention and/or the intubation aid according to the second aspect of the present invention to include a set of intermediate pieces in order to be able to provide a plurality of adapters for different tube inner diameters. Such an intermediate piece can be embodied as an overshaft that can be fitted onto the main part, for example, in the form of a metal tube.

The present invention will be described in greater detail hereinafter using an embodiment with reference to the accompanying drawings, in which:

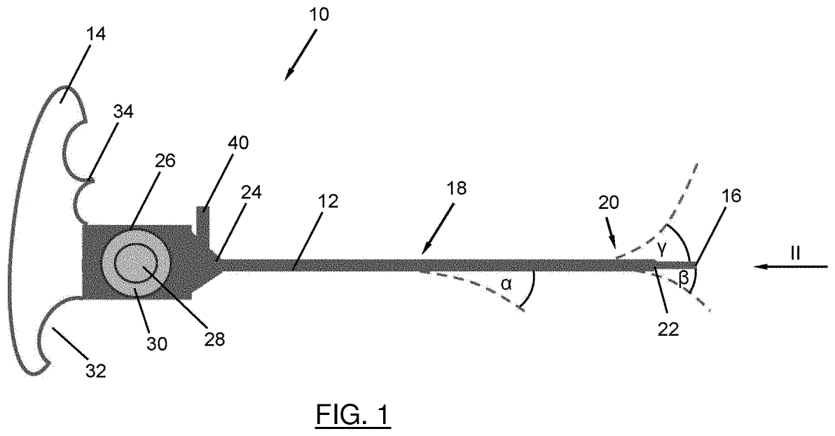

FIG. 1 shows a schematic side view of an embodiment of the inventive intubation aid according to the first aspect;

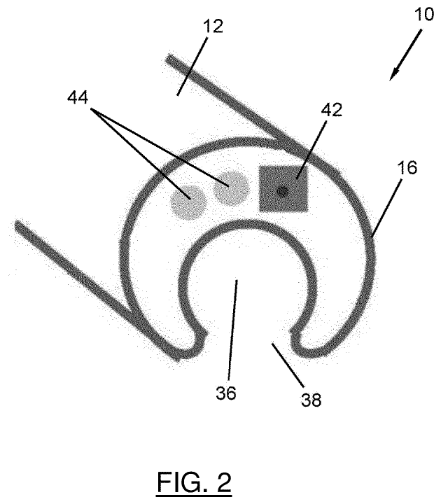

FIG. 2 shows a schematic side view of the free end of the main part of the intubation aid according to arrow II from FIG. 1;

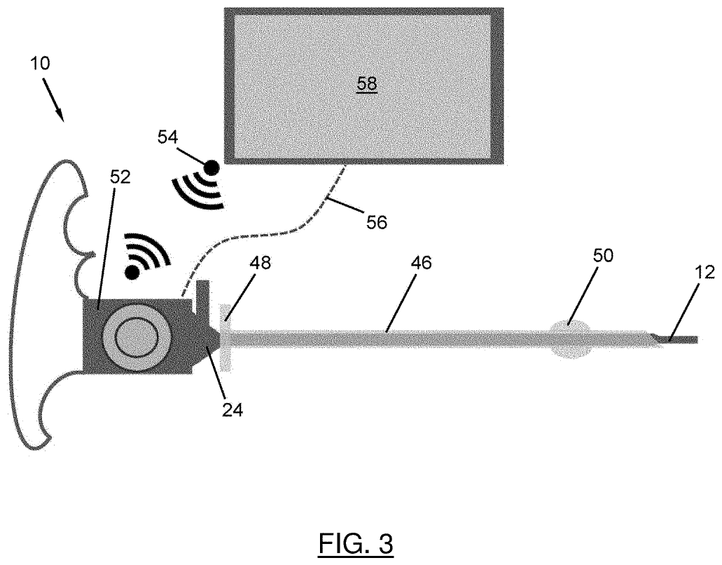

FIG. 3 shows a schematic view of the intubation aid from FIG. 1 with the tube arranged thereon and the monitor connected; and,

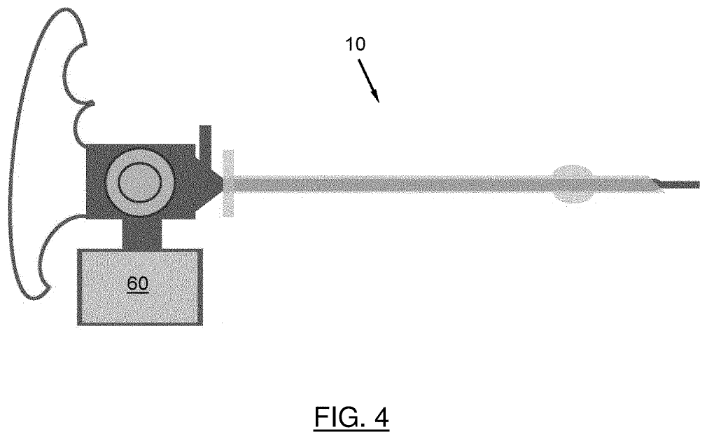

FIG. 4 shows a schematic view of the intubation aid from FIG. 1 with the tube arranged thereon and the monitor attached directly;

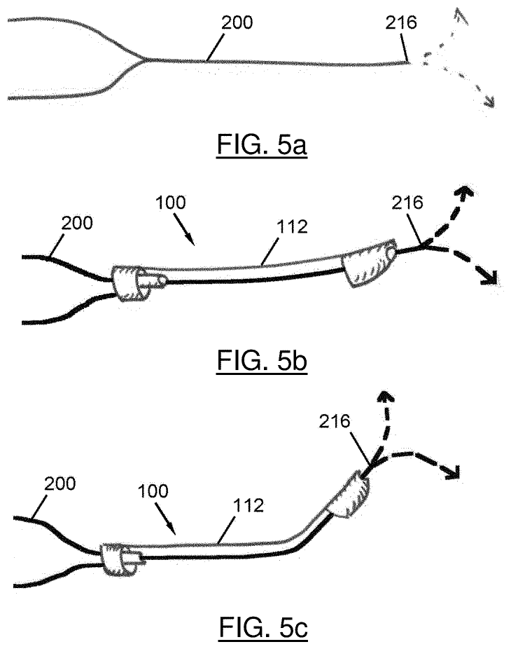

FIG. 5a shows a standard endoscope;

FIGS. 5b and 5c show the standard endoscope from FIG. 5a, on which an intubation aid according to the second aspect is arranged;

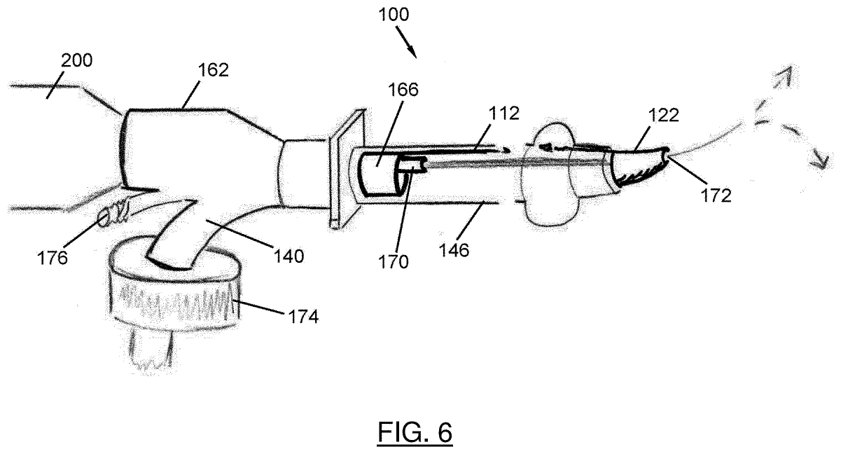

FIG. 6 shows the intubation aid according to the second aspect in a schematic sectional side view;

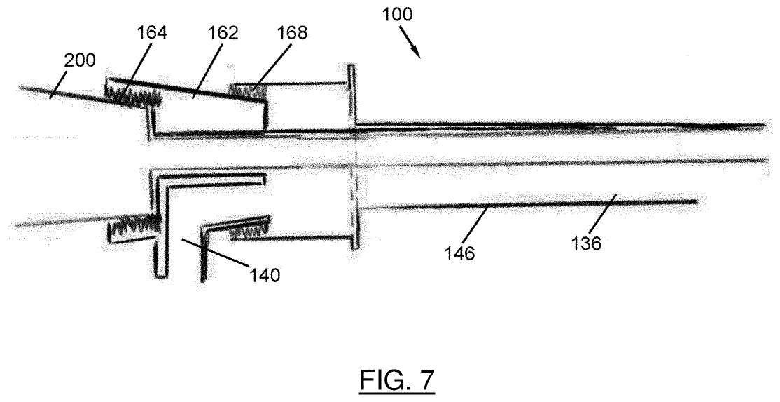

FIG. 7 shows a schematic sectional side view of the intubation aid according to the second aspect.

An inventive intubation aid is generally denoted with reference number 10 in FIG. 1. The intubation aid 10 comprises an elongate main part 12 which has a handle 14 at its one end and a free end 16 at its other end. In FIG. 1, the main part 12 is designed to be bent in a first region 18 such that a segment of the main part 12 arranged between the free end 16 and the first region 18 can be deflected downward by up to an angle α. Furthermore, the main part 12 has a second region 20, which is designed to be bent such that the free end 16 can be deflected relative to a segment between the first region 18 and the second region 20 by an angle up to 13 which extends in the same direction as the angle α, or, in the opposite direction, up to an angle γ.

Arranged between the free end 16 and the second region 20 of the main part 12 is a tapering segment 22, across which an outer diameter of the main part 12 in the segment of the second region 20 gradually decreases to an outer diameter of the main part 12 in the segment of the free end 16.

At its end opposite the free end 16, the elongate main part 12 has a positioning device 24, designed here as a cone 24. Arranged in a segment between the cone 24 and the handle 14 is an operating device 26, which comprises a first operating element 28 and a second operating element 30. The first operating element 28 is coupled to the first region 18 via a cable pull, such as a Bowden cable, for example, such that an actuation of the first operating element 28 by an operator in a corresponding direction causes a deflection of the first region 18 in accordance with the amount input on the first operating element 28. The second operating element 30 is coupled to the second region 20 in an analogous manner.

The first operating element 28 is connected to a latching device, so that a set deflection of the first region 18 can be locked using the operating element 28 itself or, for example, using a corresponding segment of the handle 14.

As can be seen in FIG. 1, the handle 14 includes a single-finger receiving element 32, which is designed in particular to receive an operator's index finger, and which is arranged on the same side of the intubation aid 10 on which the first region can be deflected by up to the angle α and the second region can be deflected by up to the angle ß, and a two-finger receiving element 34, which is designed to receive in particular the middle finger and the ring finger of the operator.

FIG. 2 shows a view of the free end 16 of the inventive intubation aid 10 from FIG. 1, as indicated by the arrow II in FIG. 1. It can be seen here that a channel 36 is arranged in the main part 12 and is formed along the longitudinal extension of the main part 12. The channel 36 is not arranged completely within the main part 12, but extends along its length, in the embodiment shown here from the free end 16 to the cone 24, via an interruption in the circumferential direction of the channel 36 with an outside of the main part 12 in fluid communication. This creates a gap 38 which, in this embodiment, extends from the free end 16 to the cone of the positioning device 24, wherein the channel 36 is accessible via this gap 38, for example for cleaning. However, as described above, it is also possible for the channel 36 to terminate at the tapering segment 22 of the main part 12.

The channel 36 is connected at its end opposite the free end 16 to an interface 40 (see FIG. 1) which is designed to be connected to a suction device and/or to a gas source and/or to a virus/bacteria filter or the like. Under the action of the suction device connected to the interface 40, aerosol can be suctioned out of the mouth/pharynx, larynx, and trachea of the patient when the patient is being intubated, so that free escape of the potentially virus-laden aerosol into an environment can be prevented. The channel 36 can also be used to introduce, for example, oxygen, into the patient from a gas source via the interface 40.

Furthermore, it can also be seen in FIG. 2 that an imaging unit 42 designed for recording imaging information in surroundings of the free end 16 of the intubation aid 10, as well as an illumination device 44, embodied here as two LED light sources 44, is embodied at the free end 16 of the main part 12.

FIG. 3 shows the intubation aid 10 from FIG. 1, wherein a ventilation tube 46 has been fitted over the main part 12. The ventilation tube 46 has a collar 48 which, after the tube 46 has been used in a patient and the intubation guide 10 has been removed from the tube 46 and the patient, functions as an interface to a ventilation device. In the region of the collar 48, the tube 46 is held by the cone 24, which acts as a holding device here, in a friction fit against a specific force that displaces the tube 46 from the main part 12 (to the right in FIG. 3). The ventilation tube 46 further has an expandable region 50 which can be filled with a gas, for example, in order to seal off an air space connected to the lungs from an air space connected to the mouth once the tube 46 is at its desired location in the patient.

The intubation aid 10 comprises a communication module 52 designed to communicate wirelessly 54 and/or in a wired connection 56 with an external unit 58, which in this case is a portable monitor, designed to display the image data recorded using the imaging unit 42.

FIG. 4 shows that a monitor 60 can be arranged directly on the intubation aid 10, as an alternative or in addition to coupling the intubation aid 10 to an external monitor according to FIG. 3, on a segment on which in particular the operating unit 26 is arranged. Of course, the intubation aid 10 can also be embodied such that it can communicate both with the monitor 58 (FIG. 3) and with the monitor 60 (FIG. 4) at the same time.

A standard endoscope 200 is shown in FIGS. 5a to 5c and has a corresponding bending mechanism at its free end 216, wherein an intubation aid 100 according to a second aspect of the present invention is attached in FIGS. 5b and 5c (of which here only one main part 112 is shown for the sake of clarity). Elements of the intubation aid 100 according to the second aspect that are similar to the intubation aid 10 according to the first aspect are provided with the same reference numbers, but increased by 100.

FIGS. 5b and 5c show two main parts 112, which each have different prebends which equate to a curvature functionality of the “first region” of the first aspect of this invention.

FIG. 6 shows a schematic side view of the intubation aid 100. It can be seen that the intubation aid 100 is fitted over the endoscope 200 until a connecting segment 162, or more precisely, in the embodiment shown, a first engagement segment 164 (see FIG. 7), engages with the endoscope 200.

A main part 112 is attached to the connecting segment 162 via a retaining clip 166 and has a tapering segment 122 at its end opposing the retaining clip 166. A ventilation tube 146 is fitted over the main part 112 such that it is held on the connecting segment 162 via a suitable second engagement segment 168 (see FIG. 7) against a releasing force which acts on the intubation aid 100 in a direction opposite to the direction in which the ventilation tube 164 is fitted on.

It can also be seen in FIG. 6 that a distal segment of the endoscope 200 is guided through receiving segments 170 and 172, respectively, of the connecting segment 162 and of the tapering segment 122.

A schematic side sectional view of the intubation aid 100 is shown in FIG. 7. As can be seen here, in the interior of the tube 146 the fitted tube 146 forms a channel 136 which can be used to suction off aerosol using a suction device connected to the interface 140 of the connecting segment 162. Going back to FIG. 6, it can be seen that in addition to the interface 140, which is connected to a virus/bacteria filter 174, an interface 176 via which oxygen can be introduced into the channel 136 is embodied separate from the interface 140 on the connecting segment 162.

1. Intubation aid (10) for inserting a tube (46) into a patient's trachea, wherein the intubation aid (10) comprises an elongate main part (12) which at its one end is connected to a handle (14) and at its other end has a free end (16), wherein the intubation aid (10), in particular the handle (14), is provided with an operating device (26), wherein the main part (12) is designed to be bent in a first region (18) of the main part (12) when the operating device (26) is actuated, wherein the main part (12) is furthermore designed, upon further actuation of the operating device (26), to be bent in a second region (20) of the main part (12), which is separate from the first region (18) and is arranged adjacent to the free end (16) of the main part (12), and wherein the main part (12) further comprises a positioning device (24) which is designed to limit a translational displacement of a tube (46) fitted onto the intubation aid (10) in at least one direction.

2. Intubation aid (10) according to claim 1, wherein the positioning device (24) comprises a holding device which is designed to engage with a segment of the tube (46) in order to secure the latter, at least translationally, relative to the main part (12), in particular wherein the holding device is a cone (24) that is arranged adjacent to the handle (14) and that engages a segment of the tube (46) in a friction fit, and/or is embodied as a locking unit which engages the segment of the tube (46) in a positive fit.

3. Intubation aid (10) according to claim 1, wherein the main part (12) comprises a channel (36) which extends parallel, at least in part, to a longitudinal axis of the main part (12) and which is suitable for receiving fluid and for conducting the fluid to an end (40) of the channel (36) assigned to the handle (14), wherein advantageously one of the following is selectively connectable to the end (40) of the channel (36) assigned to the handle (14)
a suction unit and a virus and bacteria filter suitable for filtering viruses and bacteria from the fluid suctioned out from the patient, or
a gas source, in particular an oxygen source designed to deliver oxygen to the patient via the channel (36).

4. Intubation aid (10) according to claim 3, wherein a wall of the main part (12), viewed in the circumferential direction of the elongate main part (12), is designed interrupted such that a gap (38) extending in the longitudinal direction of the main part (12) is formed and connects the channel (36) to an exterior surrounding the main part (12).

5. Intubation aid (10) according to claim 1, wherein the intubation aid (10) comprises an intermediate piece designed to be arranged between the tube (46) and the main part (12).

6. Intubation aid (10) according to claim 1, wherein the main part (12) has a tapering segment (22) which is arranged adjacent to the free end (16) of the main part (12), in particular between the free end (16) of the main part (12) and the second bendable region (20) of the main part (12).

7. Intubation aid (10) according to claim 3, wherein the channel (36) extends from the end (40) associated with the handle (14) to the tapering segment (22) of the main part (12).

8. Intubation aid (10) according to claim 1, wherein an imaging unit (42), in particular an image sensor, which is designed to record and forward imaging data, is arranged on the free end (16) of the main part (12).

9. Intubation aid (10) according to claim 1, wherein an illumination device (44), which is designed to emit light, in particular onto the region to be recorded by the imaging unit (42), is arranged at the free end (16) of the main part (12).

10. Intubation aid (10) according to claim 1, wherein the first bendable area (18) is arranged at a distance of 10 cm to 20 cm, in particular about 15 cm, from the free end (16) of the main part (12).

11. Intubation aid (10) according to claim 1, wherein planes in which the main part (12) can be curved in the first region (18) and in the second region (20) are essentially parallel to one another, in particular coinciding.

12. Intubation aid (10) according to claim 1, wherein the first region (18) can be bent from 0° to 30° and/or the second region (20) can be bent from −60° to 45°.

13. Intubation aid (10) according to claim 1, wherein the operating device (26) comprises at least a first operating element (28) and a second operating element (30), wherein the first operating element (28) is designed for deflecting the first region (18) of the main part (12) and the second operating element (30) is designed for deflecting the second region (20) of the main part (12), and wherein the first operating element (28) is assigned a latching device designed for preventing rearward movement of the first operating element (28), counter to the actuating direction thereof, upon actuation of the first operating element (28) by at least a predetermined amount.

14. Intubation aid (10) according to claim 1, wherein the intubation aid (10) comprises a communication module (52), in particular a wireless communication module (52), which is designed to communicate with an external unit (58, 60), wherein the communication module (52) is arranged in particular in the handle (14) of the intubation aid (10).

15. Intubation aid (100) for inserting a tube (46) into the trachea of a patient, comprising an elongate main part (112), a tapering segment (122) which is arranged on and connected to a longitudinal end of the main part (112), a connecting segment (162) which is arranged on and connected to the other longitudinal end of the main part (112), wherein the connecting segment (162) has a first engaging segment which in the radial direction is essentially inwardly oriented and is designed to engage with a superordinate assembly, in particular an endoscope, and has a second engagement segment which in the radial direction is essentially outwardly oriented and is designed to engage with the tube, and wherein both the connecting segment (162) and the tapering segment (122) have a receiving segment (170, 172) each, each of which is essentially embodied as a through-hole and is designed to receive a segment of the superordinate assembly, in particular the endoscope.
